(19) United States
(12) Patent Application Publication
(10) Pub. No.: US 2008/0053912 A1
(43) Pub. Date: Mar. 6, 2008

(54) Unipolar magnetic medicine carrier

(76) Inventors: Huan-Chen Li, Westford, MA (US); Wendy Wang, Westford, MA (US)

Correspondence Address: HUAN-CHEN LI, 4 SWEDES CROSSING, WESTFORD, MA, 01886, US

(21) Appl. No.: 11/526,473

(22) Filed: Sep. 25, 2006

Related U.S. Application Data
(60) Provisional application No. 60/841,653, filed on Aug. 31, 2006.

(51) Int. Cl. A61K 9/14; C02F 1/48

(52) U.S. Cl. 210/695; 424/489; 424/490; 264/5

(57) ABSTRACT

We propose unipolar magnetic particles as medicine carriers. The whole surface of each particle is monopolar, being either north or south. The particles repel each other and are repelled by same polar external magnet. Four or more magnets that are stereo located to apply the forces to a swamp of particles from all directions may, therefore, relocate the swamp, squeeze it, reshape it, and allow it to expand.

### CROSS-REFERENCE TO RELATED APPLICATION

The application claims the benefit of Provisional Application No. 60/841,653 filed on Aug. 31, 2006 for “External Magnetic Force Directed Drug Delivery”

### FIELD OF THE INVENTION

The present invention relates to use and preparation of micro and nanoparticles or beads and more particularly to unipolar magnetic or magnetizable medicine carriers.

### BACKGROUND OF THE INVENTION

The possible clinical use of magnetically guided microparticles for drug delivery to tumors and elsewhere within the body has been studied for three decades. Each microparticle is typically made up of polymers as well as many nanomagnets or ferronanomagnets that can align in a magnetic field. Once aligned, the microparticle has a north pole and a south pole, being dipolar as natural magnet does. Because of their paramagnetic or ferromagnetic feature, these microparticles are always attracted to an external magnet regardless of its polarity. Those microparticles that are in the front move faster, those in the back move slower, and those further behind may get lost, due to the external magnetic force decreases exponentially with distance. Such magnetic targeting cannot enter into hospitals for routine therapeutic uses. There are major problems associated with it.

The first is that the particles attract each other and may aggregate into a blot, hence blocking the blood flowing in the vessel and causing strokes if used in the brain, heart attacks if used in or close to the heart, and damage to other organs if under treatment. The second is that most of these particles, after the treatment, are left behind in the human body, hence causing Ferro liver failure over times. The third, which is the most fatal, is that the particles are not as maneuverable as needed for practical uses, such as you cannot concentrate a swamp of particles to the center of a tumor, you cannot reshape the swamp, you cannot resize it, and you cannot relocate it.

Although artificial unipolar magnets have been invented for decades, such as Herb's toy ball (U.S. Pat. No. 4,874,346) which is built up by many magnetic bars that point with their one same poles to the core and the other to the surface, making the whole surface unipolar, we have not found anyone prepared unipolar micro or nanoparticles, not to mention anyone ever used them.

### SUMMARY OF THE INVENTION

The present invention is about preparing as well as using unipolar magnetic micro or nanoparticles for purposes such as drug carriers.

To make the surface of the particles unipolar, we propose activate or protect one same pole end of the tiny magnets that make up part of the particles. Only the activated pole will bind to the core of a particle or to activated pole of other tiny magnets. The other pole will point to the surface, making the whole sphere surface either north or south.

To isolate the unipolar particles, we propose to apply same pole external magnetic force to the container that contains the particles to push the unipolar particles to the other end for collection.

In term of internal interactions, the unipolar particles repel each other. They will not aggregate.

Unipolar particles are always repelled by same polar external magnet. They move away from it and those moving in the front move slower and those in the back move faster, which make it easy to keep them altogether as a swamp.

They can be pushed from all directions, which make them very maneuverable. For example, a swamp of these particles carrying radioisotopes may be administered, in vivo. The swamp is immediately brought to the center of external magnetic forces that are same polar and come stereo from all directions. By adjusting the strengths of the forces, you can keep the swamp to a big size so the radiation is safe, push the swamp into the target site, squeeze it smaller so to increase the strength of the radiation to destroy the tumor, expand it to a safe size again, then relocate it for recovery.

### Preferred Embodiments

A typical unipolar particle is made of polymers, with a shell containing tiny magnets that point with one same pole to the center and the other to the surface.

Base materials such as polymers, polypeptides and polynucleotide build up the most part of a particle. The particle can be in any shape, but preferably as a sphere. The size of the particles can be any; however, we prefer them to be in the range of 1 nm to 800 micron. The size is dependent on the use, such as if we want the particles to get trapped inside a specific tumor, their size may be 1-2 micron, if we want the particles to serve as capsules for use in a gastro intestinal treatment, the size can be much bigger. The particle may have a center core. To increase the strength of the unipole magnetic force on the sphere surface, the tiny magnets that are put on the core may not get inside the center core. This means we prefer the center core to be magnet free. The center core should be as big as possible, and, possibly, filled with materials that decrease magnetic forces effectively. However, if we want the tiny magnets to be longer, they may touch each other at the center. All the tiny magnets, once installed to in the particle, point with their south poles to the center and the north poles outwards, or vise versa. We prefer the size of the tiny magnets to be 0.01 nm to 600 microns or, more preferably, 1 to 30 nm, as such sized magnets are single domain in their magnetic moments. The tiny magnets can be in any shape, such as a ball, a bar, a rod, etc. The materials that hold the magnets may also be those materials that decreases (decrease) the magnetic force effectively. We prefer to expose the polar face of the magnets to the surface of the particle. If we have to submerge the whole magnet inside the particle, for any purpose, the layer that covers the polar face of the magnets should be as thin as possible. In case we want the particle to stick onto the hydrophobic cell membrane, the outmost layer, if hydrophilic, should melt away in time so to expose any inner hydrophobic layer.

The particles may have multiple magnet layers or shells. And different layers may have same pole or different poles point outward, such as the inner layer pointing with its south pole outward and the outer layer pointing with it north pole outward, or both layer points with their north pole outward, etc.

The particles may contain or associate all known medicines, such as drugs, Boron(10), heating medium, radiation or other signal moieties. The medicines can be put inside the particle or tagged at the surface. In addition, the particles may be labeled or tagged with positrons or any other signal moieties for position detecting purposes.

In the process of manufacturing, synthesis, preparation, making or producing these particles or their components and intermediates, we propose to add a step, effort, means or procedure for the purpose of knowing, controlling, aligning or taking care of the polar direction of the tiny magnets that are to be incorporated into the particles, such as a means to let us know what direction the north poles are pointing to, etc. In another word, the old manufacturing process does not care the orientation of the tiny magnets but we do and we will have a step to take care of it, such as we may apply a strong magnetic force(s) from one or more directions to the container that contains or holds the tiny magnets. The force(s) may overcome the interactions of those magnets and make their north pole all pointing to one direction. That means we may use magnetic field to orientate the tiny magnets before, during, and after the modification of the tiny magnet and the installation of the tiny magnets to the particles.

In case of inducible magnetic material are used in place of the tiny magnets, we want the added step or means to make sure the induced magnetic force are also monopolar, Isotropic get magnetized in all direction and anisotropic magnets get installed north pole outwards or verse visa.

The added step may also take care or ensure that all the tiny magnets are installed with one same pole facing outwards and the other to the center. In all the following examples, the tiny magnets are either bare or coated with some materials.

### FIRST EXAMPLE

Put the tiny magnets against a membrane; apply some magnetic forces that are much stronger to overcome the indirections among the magnets so that they will stand with one same pole facing to the membrane. They will bind to the membrane. Then the membrane are cut into small pieces, once heated or cooled, the other face of the membrane contract to form the beads.

### SECOND EXAMPLE

In the colloid that contains the tiny magnets, we add to the top an oil or organic layer that is as deep as the length of the tiny magnets. At the top of the oil layer, we put a strong magnet to draw the tiny magnet to the layer, the other pole of the tiny magnets will stay in the solution that contains them. We may then modify the pole that is still in the solution, such as adding active groups to allow the tiny magnets to bind to the particles with that specific pole, or bind together with that modified pole then add polymers to the bound magnets.

### THIRD EXAMPLE

The particles have V shaped holes and each tiny magnet has a V shaped south pole and only that pole can get into the particle then binds there. The particles have hydrophobic surfaces and one same pole of each tiny magnet is hydrophobic and that pole can bind to the particles. These apply to chemical bounds, active groups, electric charges, enzymes and so on. This means we use those magnets that are activated at one same pole to prepare the particles. In the above way, we may protect one pole of the magnets such as coat one same pole with inner materials. In order to make one pole of the magnets special, other than the means mentioned elsewhere, we may use solid support mean such as we may use a strong magnet to absorb all the tiny magnets to the surface. The surface can be a layer of hard staff in front the magnet or just the bare surface of the magnet. In case the magnet is a ball(s) that is to be put into the solution, the surface may by unipolar. The surface can be smooth or may have many holes that are half shell. The surface may have a layer of materials such as wax or oil that may merge the selected pole and prevent the modification chemicals or means to assess the merged portion so to prevent any modification of that pole but expose the other pole to allow the modifications. We may also put some modification mechanisms such as some modification chemicals on the solid surface or in the lay of materials that are on the surface such as the above mentioned wax to modify the pole that are attracted to the solid surface of the strong magnet. The strong magnet can be electromagnetic forces as they will attract only one pole of the particles to the surface, we may treat that pole or the other pole to make either special, For example, we may dissolve the SiO2 coating or any other coating at one pole of tiny magnets that are produced by Yamamoto's method (Yamamoto, et, al Appl. Phys. Lett. 2005, 87, 032503) or remove the anionic charge at one pole of nanoparticles synthesized from Massart's method (R. Massart, IEEE Trans. Magn. 1981, 17, 1247). The other pole will not be modified such as they will still have the coating and anionic charge for the binding to the beads or core. We may use these nanoparticles for preparing medicine carriers with the method described by Dobson (United States Patent Application, publication number 2006105170 with filing date May 18, 2006). We may also modify Chen's method (U.S. Pat. No. 7,081,489) by treating only one pole with an anionic surfactant to form modified active agent nanoparticles. This means we should have a way to modify one pole to activate it for the binding. We may also protect one pole by coating or any other means as described above for activation so that the protected pole will not bind.

After the preparation, we will add another step or means to isolate the monopolar particles with the help of same polar magnetic forces. For example, we may apply a magnetic force to the medium or container that contains the particles. The magnetic force should be same polar to the surface pole of the unipole particles. The force will attract all dipole particles or tiny magnets to it and repel the unipolar particles to the other end. We may then collect the medium at the other end to harvest the unipole particles, or the particles at the other end of the container if that is the container. Such as if the medium is water, we collect the water at the other end of the container. If the magnetic force comes from the bottom, we collect the water at the top. If we merge a filter into the other end of the medium then apply the force, all unipolar particles will go to the filter and get collected. If we add a layer of another solvent at the top or bottom and then apply the force from the opposite end of the medium, the force will repel the unipolar particles into the new layer. In order to isolate top quality unipolar particles, we may add the solution, air, or other medium that contains the particles into a tube and allow the medium to flow, in the mean time, we apply a same polar magnetic force against the flow direction. Good unipolar particles will be stopped or even go against the direction of the flow due to the repelling force from the external magnet but poor ones will go along with the flow and dipolar ones will go faster than the flow speed. By collecting different fractions of the solution, we purify the unipolar magnetic particles.

Our machine can apply same polar magnetic forces stereo from all directions. Our machine allows us to apply the external magnetic forces to the particles from at least four directions, each are geometrically located in the space. They are positioned stereo-symmetrically to apply the external magnetic forces in a way that the particles receive the force of same pole magnetic forces from them as from all directions. It is obvious that, with proper adjustment of each magnetic strength, the magnetic gradient will thus create a center or focus in which the magnetic gradient is nearly zero. All external magnets face their north pole to the swamp of particles. The machine can be similar to the six-coil superconducting system using MRI technologies that generates electromagnetic forces from all directions.

A computer may control the size, shape, and location of the swamp of particles by adjusting the strength of each magnet or electromagnet. Current machines never do this. They may have many magnetic sources but they apply the force in one direction which means if the magnets on one side of the particles faces their north pole to the particles, the magnets on the other side will face their south pole to them, or vice-versa. They never face the same pole to the particles. Our machine can do it. It can even apply same strength and same pole magnetic force to the swamp from many stereo-directions at the same time so that the swamp receives the magnetic force from all directions. In the process of concentrating the swamp, our machine can apply pulsed forces, at one time, the left side sources are on while the right side sources are off, a another time, the upper side sources are one and the lower side sources are off, at still another time the right side sources are one and the left side sources are off and so on very fast intermittently. In the process of moving the swamp, such as to the right, the left side sources may have the maximum strength and all other side may be weak in order just to keep the concentration or the right side source may even be off or change to the opposite pole to for attraction in order for the swamp to move fast. Once the treatment is finished, we may move the swamp to the urine for excretion or to a location such as into a vein so that we can withdraw the particles out by a needle and syringe. Our machine has an adjusting means for adjusting the strength of each source. It also has software that may adjust the forces automatically according to the shape of the tumor. Our machine may include a sensing means that can sensor the magnetic gradient focus and/or the position of the swamp of particles. The software combined with other means will adjust the strength of each magnet to create a focus and maintain it and/or keep the swamp centered to the focus. To destroy a tumor in the brain with radioisotopes like rhenium-188 or 1-131, we may prepare tens of thousands of unipolar particles, label them with enough radioisotopes, such as 800 mci, then inject a swamp of them into the brain fluid either outside or inside the hard membrane. The swamp can also be administered orally, intravenously, through an artery, or into a local tissue. The swamp may be under the external magnetic control during the injection, and, after the injection, the swamp will be brought to the focus of the stereo-magnetic forces. The forces come from many directions in order to keep the swamp localized but big enough not to harm the surrounding tissues. The stereo-magnetic forces will then move the swamp of these particles to the tumor. During the moving, the magnets that against the moving direction may be shut off or even turned around to the opposite pole to attract the swamp, the magnets that are at the side will be kept strong, enough to keep the swamp narrow but not too narrow as to harm the surrounding tissues, the magnets that are pushing the swamp along the direction may be kept at maximum strength in order to keep the swamp short but not too short as to harm the surrounding tissues. All these magnets may be applied pulse, intermittently, or persistently. One or more controller(s) or machine(s) is in charge for the turning off and adjusting the strength and position of the magnets. Once the swamp get to the target region, the machine will turn on all magnets and apply forces from all directions to concentrate and reshape the swamp such as to the shape of the tumor. The size of the swamp can be squeezed to so small that the radiation can kill all the cells in the swamp in seconds. If in hours, we may let the particles get trapped in there through the specific size of the particles, linked there by chemical active groups, antibodies or charges, or simple keep applying the forces to keep the particles there. As cancer cells are more sensitive to radiations, we may treat the cancerous area for a predetermined time that will ensure all cancer cells get killed but normal cells will recover and survive. The length of the predetermined time is dependent on the type of cancer, the type of tissue the cancer reside in, the location of the area and many other factors. We need experiments to determine it. Once the treatment is finished, the machine will decrease the strength of the stereo-magnetic forces so to allow the swamp to expand its size in order to decrease the radiation strength, and then move the swamp to a location where the swamp can be easily withdrawn by a needle and syringe.

The above procedure may apply to the following treatments too.

Treatment 1: Thermal treatment is also very selective because cancer cells are more sensitive to heating. We may use the same procedure as the above just replace the radiation by heating-energy sensitive materials. Once the particles are concentrated into the cancerous area, we apply heating energies to heat up the particles that will, in turn, heat up that cancerous area in the swamp. In this treatment, the particles serve as medium to absorb heating energies, the particle may contain materials that get heated easily when external energies such as microwaves are applied, and the microwave length should be selected in order to preferably heat the particles over normal tissues. Currently used para or ferromagnetic particles can get heated in magnetic field. We may use the same mechanism if it is applicable to our unipolar particles.

Treatment 2: Boron neutron capture therapy is good for brain tumors. The boron(10) explosion will kill cells that are directly adjacent to it only. We may use the same procedure as the above just replace the radioisotopes with boron(10). Once the particles are concentrated into the tumor, we apply neutron beams to cause the boron(10) to explode.

Treatment 3: Photodynamic therapy, when enhanced by magnetic targeting, will be a very promising cancer treatment. Photosensitizers, such as the FDA approved photopharyn, may be carried to the cancerous region by the unipolar particles with the magnetic targeting or administered systematically to a patient. Wait for some time for the drugs to get into the cancer cells then administer luminescent labeled unipolar particles using similar procedures as the above. The photosensitizer(s) may be carried to the cancerous region with the carriers that carry the luminescent agents and at the same time. We may also first administer the particles that carry the luminescent agent then administer photosensitizers. The time for the particles to stay in the area is critical. If too long, all cells will be killed. If too short, only a minimum amount of cancer cells may be killed. We should move the particles out of the area and the body just in time. And we may need experiment to determine how long the particles should stay.

In a similar way, the particles can deliver other medicines such as enzymes, vectors, prodrugs, antibodies and chemotherapeutic agents. The particles can carry a single, a pleural or all know medicines in one single trip. The particles can release the medicines in a controlled manner. And many treatments can be carried out at the same time.

In case there are many small tumors spread in the brain or liver, we may add more external magnet sources to create multiple magnetic focuses, each control a small swamp of particles, so to have multi-microsurgeries in the above way simultaneously.

During the treatment, a camera will monitor the exact location and shape of the swamp. The image and location of the tumor should be well defined before the treatment, and, if possible, at the same time when monitoring the swamp.

This invention may also have the following potentials. As magnetic forces can even lift a million pound train, the external magnetic forces we use can be so strong that they may force the particles to go against the blood flow in the artery and veins, penetrate the vein valves, and penetrate the blood vessels, tissues, organs and organ membranes. The forces can squeeze the particles to a extreme density at the center of a tumor then suddenly loose the forces so that the particles can fly and expand outward at a speed to cause the cell to die, therefore, destroy the tumor when this process is repeated. When the forces is increased further, the particle will be in contact with each other, the tiny magnets of one particle may get inserted into the other particle which will in turn cause the particles to aggregate together, so all of them will stay to that particular location forever. The forces may be applied intermittently from different directions. The particles can release polar components at the diseased area and the polar components can be made to spin due to external forces. The spinning can kill cancer cells. As the magnetic forces can be very strong, the machine can also push and place some other devices, such as a blood vessel support means, to the heart, the brain and other organs if the device is unipolar at its surface.

We may have other embodiment that will be detailed in the future applications. Such embodiments include the followings. A procedure of manufacturing magnetic particles for medicine delivery involving a means or step for taking care of the polarity of tiny magnets that are to be installed into the particles, the tiny magnets including those that are not magnetic their own but can be magnitizable during the manufacturing time, after the manufacturing time, just before the injection, or before and during the treatment time. The machine comprises a means for moving or keeping the center of a swamp of medicine carriers in the focus and for concentrating, reshaping and relocating the swamp. And most importantly, a nano magnet which is modified differently at one pole than the other with chemical, biochemical, electronic or physical agents or groups may be patentable itself, as an intermediate. All of the above inventions may be applicable to or claimed for medical imaging such as the MRI imaging.

We claim:

1. A method of synthesizing micro or nano medicine carriers comprising:
a) a step for preparing or obtaining base materials such as polymers for building the medicine carriers;
b) a step called step M for preparing or obtaining tiny magnets being about an uniform size from the range of 0.1 nm to 500 micron;
c) a step for putting a shell of tiny magnets inside or on each medicine carrier.

2. A method according to claim 1 wherein said step M comprising a ensuring-means for ensuring one same pole of the tiny magnets get modified by some specific modification agents or both pole get modified, each with a different specific agents.

3. A method according to claim 2 wherein said ensuring-means comprising a magnetic solid surface for attracting and attaching one same pole of the tiny magnets so to prevent that pole from being accessible by modification agents in the solution.

4. A method according to claim 2 wherein said solid surface comprising a coating such as a layer of oil or wax with specific thickness for submerging a portion of the tiny magnets when they are attached to the surface in order to prevent that portion from being accessible by modification agents in the solution.

5. A method according to claim 2 wherein said solid surface being for the purpose of attracting or pushing one pole of the tiny magnets into a layer of solution leaving the other pole in a different solution, one solution having the modification agents or both having different modification agents.

6. A method according to claim 1 further comprising a means for taking care or knowing the polarity of the tiny magnets that are to be installed to the medicine carriers during the synthesis.

7. A method according to claim 6 wherein said means being a magnetic gradient applied to the solution or container containing the tiny magnets, the magnetic gradient being so strong as to overcome the internal interactions among the tiny magnets so they will orientate their poles according to the magnetic gradient.

8. A method according to claim 6 wherein said means ensuring all the installed tiny magnets pointing with their same poles to the surface of the medicine carrier, the means including only those being applicable to the said size of the tiny nano and micro magnets.

9. A method according to claim 8 wherein said means comprising using particles having functional groups to interact with one same pole of the tiny magnets for that pole to get attached to the particle, the functional groups may be V shaped holes or active chemical, biochemical, electronic and physical agents and groups.

10. A method according to claim 1 wherein said tiny magnets being identically modified at one same pole or differently modified at different poles by chemical, biochemical, electrochemical, or physical agents.

11. A method according to claim 1 further comprising an isolation process for isolating unipolar magnetic medicine carriers.

12. A method according to claim 11 wherein said isolation process further comprising:
a) applying magnetic force to a mixture that contains the medicine carriers or to a medium or container to which the mixture will be loaded at the end where the magnetic force is located, the magnetic force being the same polarity as that of the unipolar medicine carriers;
b) collecting unipolar medicine carriers from the mixture or medium at the further end of the magnetic force.

13. A method according to claim 12 wherein said mixture or medium being flowing toward said magnetic force.

14. A method according to claim 12 wherein said magnetic force being changing in strength or being moving to one end of said mixture or medium.

15. A method of magnetic field guided medicine delivery comprising:
a) administering to patient unipolar magnetic medicine carriers;
b) using external magnetic force(s) to guide the medicine carriers.

16. A method according to claim 15 wherein the magnetic force(s) being same polarity as that of the unipolar medicine carriers for pushing the medicine carriers.

17. A method according to claim 16 wherein said magnetic forces come from opposite or near opposite directions or stereo from many directions.

18. A method according to claim 15 further comprising:
a) a means to create or maintaining a center or focus of the magnetic forces and said magnetic force being nearly zero in the focus from all directions;
b) a means for put and keeping the medicine close or in the focus.

19. A machine for magnetic field guided medicine delivery comprising:
a) four or more magnets or electromagnets stereo located in a way enabling to apply same polar magnetic forces to medicine carriers from many directions, or as if from every direction in some cases;
b) a means for adjusting the strength of each magnets or electromagnets.

20. A machine according to claim 19 further comprising a means for controlling the adjusting of each magnetic forces in order to create or maintain a focus, the magnetic force from all directions being lowest or near zero in the focus and being increasing when going outwards from the focus.
